(19) United States
(12) Patent Application Publication
(10) Pub. No.: US 2004/0091428 A1
(43) Pub. Date: May 13, 2004

(54) METHOD OF PREVENTING AND TREATING MUCOSAL AND DERMAL CONDITIONS

(76) Inventor: BARRY M. LIBIN, BELLPORT, NY (US)

Correspondence Address: JAMES V COSTIGAN, HEDMAN GIBSON & COSTIGAN, 1185 AVENUE OF THE AMERICAS, NEW YORK, NY, 10036

(21) Appl. No.: 09/224,781

(22) Filed: Jan. 4, 1999

(51) Int. Cl. A61K 7/16; A61K 7/06; A61K 7/11

(52) U.S. Cl. 424/49; 424/70.11

(57) ABSTRACT

A method of treating mucositis, herpetic infections or fungal infections by contacting the affected area with an amount of composition which comprises triclosan or a combination of triclosan and a cationic antibacterial compound in an amount which is effective to alleviate the symptoms of the particular condition.

### BACKGROUND OF THE INVENTION

Many conditions arise which cause moderate to acute discomfort on the skin and mucous membranes. In particular, immunodeficient states may cause mucositis with accompanying inflammation; herpetic lesions may cause inflammation and fungal infections are also accompanied by inflammation.

Immunodeficient patients frequently exhibit a condition on the oral mucosa which is clinically described as oral mucositis. This condition has no known microbial or viral vector that has been implicated as the causative agent. The immunodeficiency that preceded the appearance of mucositis may arise spontaneously from genetic factors, may be caused by infections, e.g., the HIV virus or mucositis be induced as a result of chemotherapy or radiation therapy for neoplastic diseases. This condition has been difficult to treat and has not responded to treatment with antimicrobial or other agents.

The infections caused by Herpes simplex may appear anywhere on the skin or mucosa but are most often seen around the mouth, lips, conjunctiva, cornea and genitalia accompanied by an inflammatory response which causes pain and various degrees of discomfort. There are two existing types of herpes simplex virus infections, each type having multiple strains. Type 1 herpes simplex infects mucous membranes of the oral cavity as well as perioral skin, the skin above the waist and the eyes. A serious herpes simplex Type 1 infection is herpes keratitis which may result is disfunction of the cornea. Other primary herpes simplex Type 1 infections include stomatitis and dermatitis.

Herpes simplex Type 2 causes genital infections and is the second most common venereal disease not only in the United States but in many other countries.

It has been found that when the immune system is compromised as in the case of patients undergoing chemotherapy for cancer, the patients become highly susceptible to herpes simplex infections.

A large percentage of the U.S. population is affected by some form of a herpes virus infection, there being an estimated 98 million cases of herpes labialis (Type 1) occurring each year. In the case of genital herpes (Type 2) there are about 30 million cases each year.

Herpes simplex (Type 1) resides in latent form in the trigeminal ganglions in the facial area. In some individuals this virus remains inactive while in many others the virus may travel from the nerves located near the cheek bone to the lips. This gives rise to vesiculo-ulcerative eruptions around the lips, the chin and the cheeks, or under the nose.

Herpes simplex consists of evolving strains that are resistant to know anti-viral agents such as ganciclovir and acylovir. Because herpes simplex infections are not treatable by known antiviral agents, the usual protocol for such infections includes the elimination of the conditions which precipitated the viral infections and local antibiotic treatment to prevent to prevent bacterial at the site of the viral infection. But an antibiotic, such as penicillin is a bactericidal agent and as such is ineffective against a herpes simplex infection.

Unicellular fungi may cause dermal, mucosal or periodontal opportunistic infections. The incidence of such infections has risen with the increase in the number of immunocompromised patients, such as those suffering from HIV infections, transplant recipients treated with immunosuppressive drugs and patients undergoing chemotherapy and radiation therapy for cancer.

In the article entitled “In Vitro Antifungal Properties of Mouthrinses Containing Antimicrobial Agents” by Giulana et al., in J. Periodontal 1997; 68:791-801 it is noted that mouthrinses containing an antimicrobial agent, such as Triclosan or CPC might serve as an appropriate alternative to conventional antifungal drugs in the management of oral candidiasis.

It has been discovered that an alternative to conventional antifungal drugs is a composition in accordance with the invention in which a non-cationic antimicrobial agent combined synergistically with a cationic antimicrobial agent. This composite is also effective as a topical agent, in the form of an ointment or spray against superficial fungal infections.

This may include fungal infections of the head (tinea capitis), body infections (tinea corporis), “athletes foot” (tinea pedis) as well as groin and buttocks infections (tinea crusis) The composite has also been found to be effective against superficial candidiasis (moniliasis) and cutaneous candidiasis. Infections caused by candida are grouped under the term candidiasis and these infections involve the mucous membranes, scalp, skin and nails and may be accompanied by pain, itching and/or redness. The treatment of candida infections is discussed in MacNeill et al. J.Clin. Periodont. 24; 733-760 (1997), which is incorporated by reference.

The applicant has discovered a treatment for the inflammation which accompanies mucositis, heretic infections and fungal infections which is based on contacting the diseased sites on the affected area of the mucosa with triclosan alone or in combination with a cationic antimicrobial compound. The present inventor holds U.S. Pat. No. 5,236,699, which is incorporated by reference. That patent describes the use of a mouth rinse which contains triclosan and a cationic antimicrobial agent for use inter alia the treatment of plaque and gum diseases.

### SUMMARY OF THE INVENTION

The present invention comprises a method for the prevention or treatment of mucositis, herpetic infections and fungal infections which comprises applying to the affected area an effective amount of a composition which comprises triclosan alone or in combination with a cationic antimicrobial compound.

It is a primary object of the invention to provide a method for treating the prevention or treatment of mucositis, herpetic infections and fungal infections.

It is also an object of the invention to provide a method for treating the inflammation of oral mucositis in immunocompromised patients.

It is also an object of the invention to provide a method for treating inflammation caused by herpetic infections.

It is also an object of the invention to provide a method for treating inflammation caused by fungal infections.

It is also an object of the invention to provide a method for treating mucositis, herpetic infections or fungal infections using triclosan alone or in combination with a cationic antibacterial compound.

These and other objects of the invention will become apparent from a review of the appended specification.

### DETAILED DESCRIPTION OF THE PRESENT INVENTION

Mucositis, herpetic infections or fungal infections such as candidiasis are treated in accordance with the present invention by contacting the involved area with a composition which contains an amount of triclosan which is effective to treat the particular condition. Generally these compositions contain about 0.01 to 5.3 wt % and preferably 0.02 to 0.5 wt % of triclosan. Generally, semi-solid formulations will be formulated with higher levels of triclosan. The amount of the formulation which is applied will depend on the extent of the lesion. Generally when a liquid formulation is applied to a typical lesion, from 5 ml to 30 ml is applied to the lesion as a mouth rinse with the patient being instructed to eject the excess amount of the formulation from the mouth without swallowing. If a semi-solid formulation is used, then a thin film, i.e. from 0.5 mm to 5 mm in thickness may be applied to the affected area.

Triclosan is 2,4,4′-trichloro-2′-hydroxydiphenyl ether which is commercially available.

The triclosan is adsorbed and retained on the oral mucosa while resisting removal by saliva in the oral cavity.

The compositions may be prepared as a liquid or a semi-solid formulation. The semi-solid compositions may vary from highly viscous liquids to gels or paste like formulations.

A liquid formulation may be prepared with purified water, triclosan and a solubilizer. The solubilizer may comprise a sodium lauryl sulfate, polysorbate 20, polysorbate 40, polysorbate 60, polysorbate 80, poloxamer or mixtures thereof. The polaxamers are of the formula HO(CH₂CH₂O)ₐ(CH—(CH₃) (CH₂OH)_{b}(CH₂CH₂O)_{c}H where b is at least 15 and (CH₂CH₂O)ₐ+c is varied from 20 to 90% by weight and the weight average mol wt ranges from 10,000 to >16,000. The polyoxamers are available under the Pluronic trademark and Pluronic F127 is a preferred solubilizer. If a solubilizer is employed, it will comprise from 0.5 to 8 wt %of the liquid composition. Generally, only liquid compositions in water will require a solubilizer; semi-solid formulations will not require the presence of a solubilizer.

The mucositis treating formulation may include an anti-caries agent which is soluble in water such as sodium fluoride, stannous fluoride or sodium monofluorophosphate in an amount which is effective to inhibit tooth decay in an immunocompromised patient. Generally, this amount will be from 0.01 to 4% by weight, based on the weight of the fluoride ion. The amount may be varied depending on the particular source of the fluoride ion which is chosen. Certified color may be added in a minor amount e.g. 0.1% by weight. FD&C Blue No.1 or FD&C Yellow No.5 may be used as desired.

If a cationic antibacterial agent is used in combination with the triclosan, it may be used in combination with chlorhexidine and quaternary ammonium salts such as cetylpyridinium chloride (CPC) which is the monohydrate of the quaternary ammonium salt of pyridine and cetyl chloride. CPC is cationic, highly soluble in water and alcohol. Other cationic antimicrobial agents include benzalkonium chloride, benzethonium chloride, methylbenzethonium chloride and domiphen bromide. Chlorhexidine may be applied as the free base, or as the dihydrochloride or the gluconate salt. The composition may alternatively be based on an anionic antimicrobial agent (e.g., potassium sorbate; sodium benzoate; or methyl or propyl paraben.

The combination of triclosan and the cationic antimicrobial compound has the effect that the combined agents are readily adsorbed and retained on the oral mucosa while resisting removal by saliva in the oral cavity.

The compositions may be prepared as a liquid or a semi-solid formulation. The semi-solid compositions may vary from highly viscous liquids to gels or paste like formulations.

A liquid formulation may be prepared with purified water, the triclosan, the cationic antimicrobial compound and a solubilizer. The solubilizers have been described supra.

A typical liquid formulation will comprise:

| | % weight |
|---|---|
| triclosan | 0.100 |
| CPC | 0.024 |
| Sorbitol Solution, U.S.P. | 12.000 |
| Glycerin | 10.000 |
| Sodium Saccharin, U.S.P | 0.100 |
| Pluronic FI27, NF | 4.000 |
| 190 Proof Grain Alcohol, U.S.P. | 7.000 |
| Peppermint IFL2745 | 0.152 |
| Caramel Color AP100 | 0.0085 |
| Purified water | 66.615 |

A typical fluoridated liquid formulation will comprise:

| | % weight |
|---|---|
| triclosan | 0.100 |
| CPC | 0.024 |
| Sodium Fluoride | 0.020 |
| Sorbitol Solution, U.S.P. | 11.980 |
| Glycerin | 10.000 |
| Sodium Saccharin, U.S.P | 0.100 |
| Pluronic FI27, NF | 4.000 |
| 190 Proof Grain Alcohol, U.S.P. | 7.000 |
| Peppermint IFL2745 | 0.152 |
| Caramel Color AP100 | 0.0085 |
| Purified water | 66.615 |

A typical semisolid formulation which is a cream:

will include:

| triclosan | 0.1-5.3 | wt % |
|---|---|---|
| Cetaryl glucoside and cetaryl alcohol | 0.5-6.7 | wt % |
| (Emulgade PL 68/50, Henkel) | | |
| Cetaryl alcohol | 0.5-7.7 | wt % |
| (Lanette, Henkel) | | |
| Coco-Caprylate (Cedol LC, Henkel) | 0.5-6.0 | wt % |
| Dicapryl ether (Cetiet, Henkel) | 0.25-5.0 | wt % |
| Sweet almond oil | 0.25-5.0 | wt % |
| Petrolatum | 0.5-6.0 | wt % |
| Dimethicone (Silicone DC 200CS/Dow) | 0.1-5 | wt % |
| Phase B | | |
| CPC | 0.01-4.4 | wt % |
| glycerin | 0.5-4.6 | wt % |
| Sodium methylparaben/Sodium paraben | 0.01-0.03 | wt % |
| or | | |
| Sodium benzoate | 0.25-0.3 | wt % |
| Deionized water | 10-90 | wt % |

### An example of a semi-solid formulation according to the invention is as follows:

| Phase A | | |
|---|---|---|
| triclosan | 0.3 | wt % |
| Cetaryl glucoside and cetaryl alcohol | 3.7 | wt % |
| (Emulgade PL 68/50, Henkel) | | |
| Cetaryl alcohol | 3.7 | wt % |
| (Lanette, Henkel) | | |
| Coco-Caprylate (Cedol LC, Henkel) | 3.0 | wt % |
| Dicapryl ether (Cetiet, Henkel) | 2.0 | wt % |
| Sweet almond oil | 2.0 | wt % |
| Petrolatum | 3.0 | wt % |
| Dimethicone (Silicone DC 200CS/Dow) | 0.6 | wt % |
| Phase B | | |
| CPC | 0.1 | wt % |
| Glycerin | 2.6 | wt % |
| Sodium methylparaben | 0.18 | wt % |
| Sodium paraben | 0.02 | wt % |
| Deionized water to | 100.0 | wt % |
| Phase C | | |
| Tocopheryl acetate (cophenol 1260/Henkel) | 1.0 | wt % |

The composition is prepared by separately heating Phase A and Phase B to 80° C. prior to forming these phases. Phase C is added with stirring at 55° C. until a smooth homogeneous mixture is obtained.

The foregoing description of a preferred embodiment of the invention has been presented for purposes of illustration and description. It is not intended to be exhaustive or to limit the invention to the precise form disclosed. Obvious modifications or variations are possible in light of the above teachings. All such obvious modifications and variations are intended to be within the scope of the appended claims. A typical liquid formulation will comprise:

| | % weight |
|---|---|
| triclosan | 0.100 |
| Sorbitol Solution, U.S.P. | 12.000 |
| Glycerin | 10.000 |
| Sodium Saccharin, U.S.P | 0.100 |
| Pluronic FI27, NF | 4.000 |
| 190 Proof Grain Alcohol, U.S.P. | 7.000 |
| Peppermint IFL2745 | 0.152 |
| Caramel Color AP100 | 0.0085 |
| Purified water | 66.639 |

A typical fluoridated liquid formulation will comprise:

| | % weight |
|---|---|
| triclosan | 0.100 |
| Sodium Fluoride | 0.020 |
| Sorbitol Solution, U.S.P. | 11.980 |
| Glycerin | 10.000 |
| Sodium Saccharin, U.S.P | 0.100 |
| Pluronic FI27, NF | 4.000 |
| 190 Proof Grain Alcohol, U.S.P. | 7.000 |
| Peppermint IFL2745 | 0.152 |
| Caramel Color AP100 | 0.0085 |
| Purified water | 66.639 |

A typical semisolid formulation which is a cream:

will include:

| triclosan | 0.1-5.3 | wt % |
|---|---|---|
| Cetaryl glucoside and cetaryl alcohol | 0.5-6.7 | wt % |
| (Emulgade PL 68/50, Henkel) | | |
| Cetaryl alcohol | 0.5-7.7 | wt % |
| (Lanette, Henkel) | | |
| Coco-Caprylate (Cedol LC, Henkel) | 0.5-6.0 | wt % |
| Dicapryl ether (Cetiet, Henkel) | 0.25-5.0 | wt % |
| Sweet almond oil | 0.25-5.0 | wt % |
| Petrolatum | 0.5-6.0 | wt % |
| Dimethicone (Silicone DC 200CS/Dow) | 0.1-5 | wt % |
| Phase B | | |
| glycerin | 0.5-4.6 | wt % |
| Sodium methylparaben/Sodium paraben | 0.01-0.03 | wt % |
| or | | |
| Sodium benzoate | 0.25-0.3 | wt % |
| Deionized water | 10-90 | wt % |

An example of a semi-solid formulation according to the invention is as follows:

| Phase A | | |
|---|---|---|
| triclosan | 0.3 | wt % |
| Cetaryl glucoside and cetaryl alcohol | 3.7 | wt % |
| (Emulgade PL 68/50, Henkel) | | |
| Cetaryl alcohol | 3.7 | wt % |
| (Lanette, Henkel) | | |
| Coco-Caprylate (Cedol LC, Henkel) | 3.0 | wt % |
| Dicapryl ether (Cetiet, Henkel) | 2.0 | wt % |
| Sweet almond oil | 2.0 | wt % |
| Petrolatum | 3.0 | wt % |
| Dimethicone (Silicone DC 200CS/Dow) | 0.6 | wt % |
| Phase B | | |
| Glycerin | 2.6 | wt % |
| Sodium methylparaben | 0.18 | wt % |
| Sodium paraben | 0.02 | wt % |
| Deionized water to | 100.0 | wt % |
| Phase C | | |
| Tocopheryl acetate (cophenol 1260/Henkel) | 1.0 | wt % |

The composition is prepared by separately heating Phase A and Phase B to 80° C. prior to forming these phases. Phase C is added with stirring at 55° C. until a smooth homogeneous mixture is obtained.

The foregoing description of a preferred embodiment of the invention has been presented for purposes of illustration and description. It is not intended to be exhaustive or to limit the invention to the precise form disclosed. Obvious modifications or variations are possible in light of the above teachings. All such obvious modifications and variations are intended to be within the scope of the appended claims.

I claim:

1. A method of treating mucositis, herpetic infections or fungal infections, said method comprising contacting the affected diseased sites with an effective amount of a composition which comprises triclosan or a combination of triclosan and an antimicrobial compound.

2. A method of treating mucositis in an immunocompromised patient, said method comprising contacting the affected diseased sites with an amount of composition which comprises triclosan which is effective to alleviate the symptoms of mucositis.

3. A method of treating mucositis as defined in claim 2 wherein the triclosan is combined in a liquid formulation with a solubilizer.

4. A method of treating mucositis as defined in claim 2 wherein the triclosan is in a semi-solid formulation.

5. A method of treating mucositis in an immunocompromised patient, said method comprising contacting the affected area with an amount of composition which consists essentially of triclosan in an amount which is effective to alleviate the symptoms of mucositis.

6. A method of treating mucositis in an immunocompromised patient as defined in claim 2 wherein the composition includes a fluoride.

7. A composition for treating mucositis in an immunocompromised patient, said composition comprising a composition which comprises triclosan in an amount which is effective to alleviate the symptoms of mucositis and an amount of a fluoride compound which is effective to inhibit tooth decay in an immunocompromised patient.

8. A method of treating mucositis in an immunocompromised patient, said method comprising contacting the affected diseased sites with an amount of composition which comprises triclosan and a cationic antibacterial agent in amounts which are effective to alleviate the symptoms of mucositis.

9. A method of treating mucositis as defined in claim 7 wherein the cationic agent is selected from the group consisting of chlorhexidine, cetylpyridium chloride, benzalkonium chloride, benzethonium chloride, methylbenzethonium chloride and domiphen bromide.

10. A method of treating mucositis as defined in claim 7 wherein the cationic agent is cetylpyridium chloride.

11. A method of treating mucositis as defined in claim 10 wherein the triclosan and cationic agent are combined in a liquid formulation with a solubilizer.

12. A method of treating mucositis as defined in claim 10 wherein the triclosan and the cationic agent are combined in a semi-solid formulation.

13. A method of treating mucositis in an immunocompromised patient, said method comprising contacting the affected area with an amount of composition which consists essentially of triclosan and a cationic antimicrobial agent in amounts which are effective to alleviate the symptoms of mucositis.

14. A method of treating mucositis in an immunocompromised patient wherein the composition includes a fluoride.

15. A method of treating a herpetic infection, said method comprising contacting the affected diseased sites with an amount of composition which comprises triclosan which is effective to alleviate the symptoms of the herpetic infections.

16. A method of treating mucositis as defined in claim 15 wherein the triclosan is combined in a liquid formulation with a solubilizer.

17. A method of treating mucositis as defined in claim 15 wherein the triclosan is in a semi-solid formulation.

18. A method of treating herpetic infections said method comprising contacting the affected diseased sites with an amount of composition which comprises triclosan and a cationic antimicrobial agent in amounts which are effective to alleviate the symptoms of a herpetic infection.

19. A method of treating herpetic infections as defined in claim 18 wherein the cationic agent is selected from the group consisting of chlorhexidine, cetylpyridium chloride, benzalkonium chloride, benzethoniumchloride, methylbenzethonium chloride and domiphen bromide.

20. A method of treating herpetic infections as defined in claim 19 wherein the cationic agent is cetylpyridium chloride.

21. A method of treating herpetic infections as defined in claim 10 wherein the triclosan and cationic agent are combined in a liquid formulation with a solubilizer.

22. A method of treating herpetic infections as defined in claim 20 wherein the triclosan and the cationic agent are combined in a semi-solid formulation.

23. A method of treating fungal infections, said method comprising contacting the affected diseased sites with an amount of composition which comprises triclosan which is effective to alleviate the symptoms of said fungal infection.

24. A method of treating fungal infections as defined in claim 23 wherein the triclosan is combined in a liquid formulation with a solubilizer.

25. A method of treating fungal infections as defined in claim 23 wherein the triclosan is in a semi-solid formulation.

26. A method of treating fungal infections said method comprising contacting the affected diseased sites with an amount of composition which comprises triclosan and a cationic antimicrobial agent in amounts which are effective to alleviate the symptoms of said fungal infections.

27. A method of treating fungal infections as defined in claim 26 wherein the cationic agent is selected from the group consisting of chlorhexidine, cetylpyridium chloride, benzalkonium chloride, benzethoniumchloride, methylbenzethonium chloride and domiphen bromide.

28. A method of treating fungal infections as defined in claim 26 wherein the cationic agent is cetylpyridium chloride.

29. A method of treating fungal infections as defined in claim 26 wherein the triclosan and cationic agent are combined in a liquid formulation with a solubilizer.

30. A method of treating fungal infections as defined in claim 26 wherein the triclosan and the cationic agent are combined in a semi-solid formulation.
